SEQUENCE LISTING (52 sequences)

SEQ ID NO: 1 - DNA, 30 bp, Artificial Sequence
  Primer xyN
ccggaattcatggtcggctttacccccgtt

SEQ ID NO: 2 - DNA, 42 bp, Artificial Sequence
  Primer XyC
aaggaaaaaagcggccgcactagtttagcccacgtcagcaac

SEQ ID NO: 3 - DNA, 678 bp, Artificial Sequence
  Synthetic
atggttggttttactccagttgctttagctgctttagctgctactggtgctttagctttcccagcaggtaatgctactgaattagaaaaaagacaaacaactccaaattctgaaggttggcatgatggttattattattcttggtggtctgatggtggtgctcaagctacttatactaatttagaaggtggtacctatgaaatttcttggggtgatggtggtaatttagttggtggtaaaggttggaatccaggtttaaatgcaagagctattcattttgaaggtgtttatcaaccaaatggtaattcttatttagctgtttatggttggactagaaatccattagttgaatattatattgttgaaaattttggtacttatgatccatcttctggtgctactgatttaggtactgttgaatgtgatggttctatttatagattaggtaaaactactagagttaatgcaccatctattgatggtactcaaactttcgatcaatattggtctgttagacaagataaaagaacttctggtactgttcaaactggttgtcatttcgatgcttgggctagagctggtttgaatgttaatggtgatcattattatcaaattgttgcaactgaaggttatttctcttctggttatgctagaattactgttgctgatgttggttaa

SEQ ID NO: 4 - DNA, 120 bp, Artificial Sequence
  Primer XyCl
atggttggttttactccagttgctttagctgctttagctgctactggtgctttagctttcccagcaggtaatgctactgaattagaaaaaagacaaacaactccaaattctgaaggttgg

SEQ ID NO: 5 - DNA, 100 bp, Artificial Sequence
  Primer XyC2r
cataggtaccaccttctaaattagtataagtagcttgagcaccaccatcagaccaccaagaataataataaccatcatgccaaccttcagaatttggaga

SEQ ID NO: 6 - DNA, 120 bp, Artificial Sequence
  Primer XyC3
ggtacctatgaaatttcttggggtgatggtggtaatttagttggtggtaaaggttggaatccaggtttaaatgcaagagctattcattttgaaggtgtttatcaaccaaatggtaattct

SEQ ID NO: 7 - DNA, 120 bp, Artificial Sequence
  Primer XyC4r
ctagtagttttacctaatctataaatagaaccatcacattcaacagtacctaaatcagtagcaccagaagatggatcataagtaccaaaattttcaacaatataatattcaactaatgga

SEQ ID NO: 8 - DNA, 120 bp, Artificial Sequence
  Primer xyC5
ttctggtgctactgatttaggtactgttgaatgtgatggttctatttatagattaggtaaaactactagagttaatgcaccatctattgatggtactcaaactttcgatcaatattggtc

SEQ ID NO: 9 - DNA, 120 bp, Artificial Sequence
  Primer xyC6r
taataatgatcaccattaacattcaaaccagctctagcccaagcatcgaaatgacaaccagtttgaacagtaccagaagttcttttatcttgtctaacagaccaatattgatcgaaagtt

SEQ ID NO: 10 - DNA, 89 bp, Artificial Sequence
  Primer xyC7
ttaatggtgatcattattatcaaattgttgcaactgaaggttatttctcttctggttatgctagaattactgttgctgatgttggttaa

SEQ ID NO: 11 - DNA, 36 bp, Artificial Sequence
  Primer xyc12N
ataagaatgcggccgcatggttggttttactccagt

SEQ ID NO: 12 - DNA, 30 bp, Artificial Sequence
  Primer xyc12C
tcataggtaccaccttctaaattagtataa

SEQ ID NO: 13 - DNA, 36 bp, Artificial Sequence
  Primer xyc37N
ttagaaggtggtacctatgaaatttcttggggtgat

SEQ ID NO: 14 - DNA, 36 bp, Artificial Sequence
  Primer xyc37C
ttttccttttgcggccgcttaaccaacatcagcaac

SEQ ID NO: 15 - DNA, 33 bp, Artificial Sequence
  Primer a)
ccggaattcatggttggttttactccagttgct

SEQ ID NO: 16 - DNA, 32 bp, Artificial Sequence
  Primer b)
ggactagtttaaccaacatcagcaacagtaat

SEQ ID NO: 17 - DNA, 89 bp, Artificial Sequence
  Primer A1
ccggaattcatgattactctgactttcctgctttcggcggcgtatctgctttctggtagagtgtctgcggcacctagttctgctggctc

SEQ ID NO: 18 - DNA, 35 bp, Artificial Sequence
  Primer B1
ctagactagtctagtcaactaaagcactctcccca

SEQ ID NO: 19 - DNA, 1398 bp, Artificial Sequence
  Synthetic
atgattactttaactttcttattatcagctgcttatttattatctggtagagtttctgctgccccttcttctgctggttccaagtcctgtgatactgttgacttaggttaccaatgttcccctgctacttctcatctatggggtttatactcaccattcttttcattagaagacgaattatccgtttcatctaagttaccaaaggattgtagaatcaccttggttcaagttctatcaagacatggtgctagatacccaacctcctccaagtccaaaaagtataagaagttagttactgctatccaagccaacgccaccgacttcaagggtaagtttgcctttttgaagacttacaactatactttaggtgctgatgacttaactccatttggtgaacaacaattagttaactcaggtatcaagttctaccaaaggtacaaggctttagctagatctgttgttccatttattagagcctcaggttcagacagagttattgcttcaggtgaaaagttcatcgaaggtttccaacaagctaagttagctgatcctggtgctactaacagagccgctccagctatttctgttattattccagaatccgaaactttcaacaacactttagaccacggtgtttgtactaagtttgaagcttctcaattaggtgatgaagttgctgccaacttcactgctttatttgccccagacatcagagctagagccgaaaagcatttacctggtgttactttaacagacgaagacgtcgtttctctaatggacatgtgttcatttgatactgttgctagaacctccgacgcctctcaattatcaccattctgtcaattattcactcacaacgaatggaagaagtacaactacttacaatccttgggtaagtactacggttacggtgccggtaaccctttaggtccagctcaaggtattggtttcaccaacgaattaattgccagattgactcgttcaccagttcaagaccacacctccactaactcaactctagtctccaacccagccaccttcccattgaacgctaccatgtacgtcgacttttcacacgacaactccatggtttccatcttctttgccttgggtttatacaacggtactgaaccattgtccagaacctcagttgaatccgccaaggaattggatggttattctgcctcctgggttgttcctttcggtgctagagcctacttcgaaactatgcaatgtaagtcagaaaaggaacctttagttagagctttgattaacgacagagttgttccattacatggttgtgatgttgacaagttaggtagatgtaagttaaacgactttgtcaagggtttgtcttgggccagatctggtggtaactggggtgaatgtttttcttga

SEQ ID NO: 20 - DNA, 40 bp, Artificial Sequence
  Primer phy1
atgattactttaactttcttattatcagctgcttatttat

SEQ ID NO: 21 - DNA, 40 bp, Artificial Sequence
  Primer phy2r
agcagaaactctaccagataataaataagcagctgataat

SEQ ID NO: 22 - DNA, 40 bp, Artificial Sequence
  Primer phy3
tatctggtagagtttctgctgccccttcttctgctggttc

SEQ ID NO: 23 - DNA, 40 bp, Artificial Sequence
  Primer phy4r
caacagtatcacaggacttggaaccagcagaagaaggggc

SEQ ID NO: 24 - DNA, 40 bp, Artificial Sequence
  Primer phy5
caagtcctgtgatactgttgacttaggttaccaatgttcc

SEQ ID NO: 25 - DNA, 40 bp, Artificial Sequence
  Primer phy6r
catagatgagaagtagcaggggaacattggtaacctaagt

SEQ ID NO: 26 - DNA, 40 bp, Artificial Sequence
  Primer phy7
cctgctacttctcatctatggggtttatactcaccattct

SEQ ID NO: 27 - DNA, 40 bp, Artificial Sequence
  Primer
taattcgtcttctaatgaaaagaatggtgagtataaaccc

SEQ ID NO: 28 - DNA, 40 bp, Artificial Sequence
  Primer phy9
tttcattagaagacgaattatccgtttcatctaagttacc

SEQ ID NO: 29 - DNA, 40 bp, Artificial Sequence
  Primer phy10r
aggtgattctacaatcctttggtaacttagatgaaacgga

SEQ ID NO: 30 - DNA, 40 bp, Artificial Sequence
  Primer phy11
aaaggattgtagaatcaccttggttcaagttctatcaaga

SEQ ID NO: 31 - DNA, 40 bp, Artificial Sequence
  Primer phy12r
gttgggtatctagcaccatgtcttgatagaacttgaacca

SEQ ID NO: 32 - DNA, 40 bp, Artificial Sequence
  Primer phy13
catggtgctagatacccaacctcctccaagtccaaaaagt

SEQ ID NO: 33 - DNA, 40 bp, Artificial Sequence
  Primer phy14r
agcagtaactaacttcttatactttttggacttggaggag

SEQ ID NO: 34 - DNA, 40 bp, Artificial Sequence
  Primer phy15
ataagaagttagttactgctatccaagccaacgccaccga

SEQ ID NO: 35 - DNA, 40 bp, Artificial Sequence
  Primer phy16r
aggcaaacttacccttgaagtcggtggcgttggcttggat

SEQ ID NO: 36 - DNA, 40 bp, Artificial Sequence
  Primer phy17
cttcaagggtaagtttgcctttttgaagacttacaactat

SEQ ID NO: 37 - DNA, 40 bp, Artificial Sequence
  Primer phy18r
aagtcatcagcacctaaagtatagttgtaagtcttcaaaa

SEQ ID NO: 38 - DNA, 40 bp, Artificial Sequence
  Primer phy19
actttaggtgctgatgacttaactccatttggtgaacaac

SEQ ID NO: 39 - DNA, 40 bp, Artificial Sequence
  Primer phy20r
gatacctgagttaactaattgttgttcaccaaatggagtt

SEQ ID NO: 40 - DNA, 40 bp, Artificial Sequence
  Phy 21
aattagttaactcaggtatcaagttctaccaaaggtacaa

SEQ ID NO: 41 - DNA, 40 bp, Artificial Sequence
  Primer phy23
caacagatctagctaaagccttgtacctttggtagaactt

SEQ ID NO: 42 - DNA, 40 bp, Artificial Sequence
  Primer phy23
ggctttagctagatctgttgttccatttattagagcctca

SEQ ID NO: 43 - DNA, 40 bp, Artificial Sequence
  Primer phy24
gcaataactctgtctgaacctgaggctctaataaatggaa

SEQ ID NO: 44 - DNA, 40 bp, Artificial Sequence
  Primer Phy26r
ggttcagacagagttattgcttcaggtgaaaagttcatcg

SEQ ID NO: 45 - DNA, 40 bp, Artificial Sequence
  Primer phy26r
cttagcttgttggaaaccttcgatgaacttttcacctgaa

SEQ ID NO: 46 - DNA, 40 bp, Artificial Sequence
  Primer phy27
aaggtttccaacaagctaagttagctgatcctggtgctac

SEQ ID NO: 47 - DNA, 40 bp, Artificial Sequence
  Primer phy28r
tagctggagcggctctgttagtagcaccaggatcagctaa

SEQ ID NO: 48 - DNA, 40 bp, Artificial Sequence
  Primer phy29
taacagagccgctccagctatttctgttattattccagaa

SEQ ID NO: 49 - DNA, 40 bp, Artificial Sequence
  Primer phy30r
gtgttgttgaaagtttcggattctggaataataacagaaa

SEQ ID NO: 50 - DNA, 40 bp, Artificial Sequence
  Primer phy31
tccgaaactttcaacaacactttagaccacggtgtttgta

SEQ ID NO: 51 - DNA, 40 bp, Artificial Sequence
  Primer phy32r
ttgagaagcttcaaacttagtacaaacaccgtggtctaaa

SEQ ID NO: 52 - DNA, 40 bp, Artificial Sequence
  Pirmer phy33
ctaagtttgaagcttctcaattaggtgatgaagttgctgc

(19) United States
(12) Patent Application Publication
(10) Pub. No.: US 2003/0022280 A1
(43) Pub. Date: Jan. 30, 2003

(54) Compositions and methods for producing high yields of heterologous polypeptides in a pichia cell

(75) Inventors: Shinobu Takagi, Funabashi-shi (JP); Yuji Terui, Chiba-shi (JP); Noriko Tsutsumi, Ichikawa-shi (JP); Rikako Taira, Tokyo (JP)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/803,454

(22) Filed: Mar. 9, 2001

Related U.S. Application Data
(60) Provisional application No. 60/190,441, filed on Mar. 17, 2000.

(30) Foreign Application Priority Data
Mar. 10, 2000 (DK) PA 2000 00392

(51) Int. Cl. C12P 21/02; C12N 1/18; C12N 15/74

(52) U.S. Cl. 435/69.1; 435/254.23; 435/483

(57) ABSTRACT

The present invention relates to compositions and methods for producing high yields of heterologous polypeptides in a Pichia cell such as Pichia methanolica are of interest.

### CROSS-REFERENCE TO RELATED APPLICATIONS

This application claims priority of Danish application nos. PA 2000 00392 filed Mar. 10, 2000 and PA2000 00419 filed Mar. 15, 2000 and of U.S. provisional application No. 60/190,441 filed Mar. 17, 2001, the contents of which are fully incorporated herein by reference.

### FIELD OF INVENTION

The high yield production of certain polypeptides is of industrial importance, thus compositions and methods for producing high yields of heterologous polypeptides in a Pichia cell such as Pichia methanolica are of interest.

### BACKGROUND OF THE INVENTION

Yeast cells are potential candidates for host organisms for heterologous protein production. To use Saccharomyces cerevisiae for the production of e.g. pharmaceutical products or industrial enzymes is well known. It has been suggested that methylotrophic yeasts that can utilize methanol as a sole carbon and energy source are more attractive host strains for heterologous protein production. High protein productivity has been achieved with strongly inducible promoters, such as the one from alcohol oxidase or formate dehydrogenase, by performing high cell density fermentations in which the dried cell mass exceeds 100 g/kg. Examples of high expression of heterologous protein are: 2.5 g/l of S. cerevisiae invertase (Tschopp et. al. Bio/Technology, vol 5, 1305-1308, 1987); 3.4 g/l of human serum albumin in Pichia pastoris (EP 510693); 3.4 g/l of glucoamylase from Rhizopus oryzae in Candida boidinii (Sakai et.al, Biochem. Biophys. Acta, 1308, 81-87,1996); 1.4 g/l of Schawanniomyces occidentalis glucoamylase (Gelissen et.al, Bio/Technology, vol. 9, 291-295, 1991); and 7.2 g/l of Aspergillus phytase in Hansenula polymorpha (EP911416). In WO 99/67398 an Aspergillus niger phytase is expressed in and secreted from Pichia pastoris by using a fusion with the signal peptide of yeast α-factor.

Pichia methanolica is another methylotrophic yeast that has been shown recently to produce human glutamate decarboxylase at the level of 0.5 g/l intracellularly (Yeast, 14, 11-23, 1998). It has been stated that P. methanolica could be used for production of any heterologous protein, but no example of productivity or yield over 1 gram per liter has been shown yet (U.S. Pat. No. 5,716,808; WO 97/17450).

### SUMMARY OF THE INVENTION

The problem to be solved by this invention is to provide expression systems and methods for producing a polypeptide of interest in high yields.

The solution is based on that the present inventors have identified compositions and methods for high yield production of a heterologous polypeptide of interest in a Pichia cell, specifically Pichia methanolica. By adjusting the codon usage of a gene encoding a polypeptide of interest to the codon usage of one or more gene(s) from the wild type P. methanolica, the inventors have shown that protein productivity surprisingly can be increased with as much as a factor of 10 or more.

In non-limiting example herein heterologous polypeptides were expressed from codon-optimized genes with original secretion signal sequences and secreted from P. methanolica.

In the industrial production of polypeptides, a gene encoding a polypeptide of interest is most often present within the production host cell in multiple copies which are all expressed in order to achieve an optimally high yield of the polypeptide. Gene analysis by Southern blotting of a production cell of the invention revealed that the high polypeptide yield was achieved with a very low gene dosage of only approx. 2 copies pr. cell, which suggests a potential for increasing the yield of the polypeptide of interest even further by increasing the copy number.

Accordingly in a first aspect, the present invention relates to a Pichia cell comprising at least one copy of a heterologous nucleotide sequence encoding a polypeptide of interest, wherein the codon usage of said sequence has been adjusted to match the preferred codon usage of P. methanolica, as defined herein.

The preferred codon usage of P. methanolica as relates to this invention is presented in Table 1 below, e.g. as the codon-ratios used in the optimized phytase gene sequence for each amino acid. More specifically the preferred codons for Lysine are TTA or TTG; for Isoleucine they are ATT or ATC; for Valine they are GTT or less preferred GTC; for Serine they are TCT, TCC or TCA; for Proline they are CCA or less preferred CCT; for Threonine they are ACT or less preferred ACC; for Alanine they are GCT or GCC; for Tyrosine the more preferred is TAC; for Glutamine it is CAA; for Asparagine it is AAC; for Glutamic acid it is GAA; for Cysteine it is TGT; for Arginine it is AGA; and finally for Glycine it is GGT.

The codon usage of a DNA sequence encoding a polypeptide is adjusted to match the preferred codon usage of P. methanolica when at least 50% of the codons for specific amino acids in the polypeptide sequence have been adjusted to a preferred codon as defined above, or preferably at least 60%, more preferably at least 70%, even more preferably 80%, yet more preferably 90%, and most preferably 95% of the codons for specific amino acids in the polypeptide sequence have been adjusted to a preferred codon as defined above.

Further in a second aspect, the present invention relates to an isolated DNA construct comprising at least one copy of a nucleotide sequence encoding a polypeptide heterologous to Pichia methanolica, wherein the codon usage of said sequence has been adjusted to match the preferred codon usage of P. methanolica, as defined herein.

Finally in a third aspect the present invention relates to a method of producing a polypeptide of interest in a Pichia cell, where the polypeptide is encoded by a nucleotide sequence heterologous to P. methanolica, said method comprising the steps of:

(a) adjusting the codon usage of the sequence to match the preferred codon usage of P. methanolica, as defined herein; and

(b) cultivating a Pichia cell comprising at least one copy of the codon usage adjusted sequence of step (a) under appropriate growth conditions to express the sequence and achieve production of the polypeptide of interest.

Deposited Microorganisms

The entire sequence of the synthetic codon optimized A. fumigatus phytase gene (SEQ ID NO:19) is comprised in the strain E. coli NN049526, which was deposited with “Deutsche Sammlung von Mikroorganismen und Zellkulturen GmbH” (DSMZ) Mar. 1, 2000 with the accession number DSM 13352.

The cDNA sequence of the Thermomyces lanuginosus DSM4109 xylanase gene was published in WO 96/32472. For this study we obtained a Saccharomyces cerevisiae strain from DSMZ, accession number DSM 10133, deposited on Jul. 19, 1995, which contains a plasmid comprising the full-length xylanase cDNA of the Thermomyces lanuginosus DSM4109.

The deposits were made by Novo Nordisk A/S and were later assigned to Novozymes A/S.

Definitions

The term “isolated nucleic acid sequence” as used herein refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis.

For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the polypeptide, insertion of the fragment into a vector molecule, and incorporation of the recombinant vector into a host cell where multiple copies or clones of the nucleic acid sequence will be replicated.

The nucleic acid sequences of the present invention may be of genomic, cDNA, RNA, semisynthetic, synthetic origin, or any combinations thereof and may be obtained from microorganisms of any genus. For purposes of the present invention, the term “obtained from” as used herein in connection with a given source shall mean that the polypeptide encoded by the nucleic acid sequence is produced by the source or by a cell in which the nucleic acid sequence from the source has been inserted.

The nucleic acid sequences may be obtained from a bacterial source. For example, these polypeptides may be obtained from a gram positive bacterium such as a Bacillus strain, e.g., Bacillus alkalophilus, Bacillus amyloliquefaciens, Bacillus brevis, Bacillus circulars, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

The nucleic acid sequences may be obtained from a fungal source, and more preferably from a yeast strain such as a Candida, Hansenula, Kluyveromyces, Pichia, Saccharomyces, Schizosaccharomyces, or Yarrowia strain; or more preferably from a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain.

The nucleic acid sequences may thus be obtained from Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis; or indeed from from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

In a preferred embodiment, the nucleic acid sequence is the sequence encoding a phytase comprised in Escherichia coli DSM13352. In another preferred embodiment, the nucleic acid sequence is SEQ ID NO:3 or SEQ ID NO:19.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents. Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

Furthermore, such nucleic acid sequences may be identified and obtained from other sources including microorganisms isolated from nature (e.g., soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding a polypeptide has been detected with the probe(s), the sequence may be isolated or cloned by utilizing techniques which are known to those of ordinary skill in the art (see, e.g., Sambrook et al., 1989,supra).

The techniques used to isolate or clone a nucleic acid sequence encoding a polypeptide are known in the art and include isolation from genomic DNA, preparation from cDNA, or a combination thereof. The cloning of the nucleic acid sequences of the present invention from such genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used.

A nucleic acid sequence of the present invention may also encode fused polypeptides or cleavable fusion polypeptides in which another polypeptide is fused at the N-terminus or the C-terminus of the polypeptide or fragment thereof. A fused polypeptide is produced by fusing a nucleic acid sequence (or a portion thereof) encoding another polypeptide to a nucleic acid sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

The present invention also relates to nucleic acid constructs comprising a nucleic acid sequence of the present invention operably linked to one or more control sequences which direct the expression of the coding sequence in a suitable host cell under conditions compatible with the control sequences. Expression will be understood to include any step involved in the production of the polypeptide including, but not limited to, transcription, post-transcriptional modification, translation, post-translational modification, and secretion.

“Nucleic acid construct” or “DNA construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term expression cassette when the nucleic acid construct contains all the control sequences required for expression of a coding sequence of the present invention. The term “coding sequence” is defined herein as a portion of a nucleic acid sequence which directly specifies the amino acid sequence of its protein product. The boundaries of the coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An isolated nucleic acid sequence encoding a polypeptide of the present invention may be manipulated in a variety of ways to provide for expression of the polypeptide. Manipulation of the nucleic acid sequence prior to its insertion into a vector may be desirable or necessary depending on the expression vector. The techniques for modifying nucleic acid sequences utilizing recombinant DNA methods are well known in the art.

The term “control sequences” is defined herein to include all components which are necessary or advantageous for the expression of a polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide. The term “operably linked” is defined herein as a configuration in which a control sequence is appropriately placed at a position relative to the coding sequence of the DNA sequence such that the control sequence directs the expression of a polypeptide.

The control sequence may be an appropriate promoter sequence, a nucleic acid sequence which is recognized by a host cell for expression of the nucleic acid sequence. The promoter sequence contains transcriptional control sequences which mediate the expression of the polypeptide. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, Yeast 8: 423-488.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound.

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the polypeptide at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids.

The vector may be an autonomously replicating vector, i.e., a vector which exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like.

Suitable markers for yeast host cells are ADE1, ADE2, HIS3, LEU2, LYS2, MET3, TRP1, and URA3.

The vectors of the present invention preferably contain an element(s) that permits stable integration of the vector into the host cell genome or autonomous replication of the vector in the cell independent of the genome of the cell. For integration into the host cell genome, the vector may rely on the nucleic acid sequence encoding the polypeptide or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of origins of replication for use in a yeast host cell are the two micron origins of replication, ARS 1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75: 1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

The present invention also relates to recombinant host cells, comprising a nucleic acid sequence of the invention, which are advantageously used in the recombinant production of the polypeptides. A vector comprising a nucleic acid sequence of the present invention is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

The present invention also relates to methods for producing a polypeptide comprising cultivating a host cell under conditions suitable for production of the polypeptide. In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptides may be detected using methods known in the art that are specific for the polypeptides. These detection methods may include use of specific antibodies, formation of an enzyme product, or disappearance of an enzyme substrate. For example, an enzyme assay may be used to determine the activity of the polypeptide as described herein.

The resulting polypeptide may be recovered by methods known in the art. For example, the polypeptide may be recovered from the nutrient medium by conventional procedures including, but not limited to, centrifugation, filtration, extraction, spray-drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J. -C. Janson and Lars Ryden, editors, VCH Publishers, New York, 1989).

### DETAILED DESCRIPTION OF THE INVENTION

A Pichia cell comprising at least one copy of a heterologous nucleotide sequence encoding a polypeptide of interest, wherein the codon usage of said sequence has been adjusted to match the preferred codon usage of P. methanolica, as defined herein.

The specific Pichia cell may be any Pichia cell, such as a Pichia methanolica, Pichia pastoris, Pichia anomala, Pichia stipitis, Pichia angusta or Pichia guilliermondii cell. Preferably the specific Pichia cell is a Pichia methanolica cell.

A preferred embodiment of the invention relates to a Pichia cell of the first aspect of the invention, wherein the nucleotide sequence encodes an enzyme, preferably the nucleotide sequence encodes an enzyme having phytase or xylanase activity, or even more preferably the nucleotide sequence comprises a DNA sequence at least 90% identical, or preferably at least 93% identical, or more preferably at least 95% identical, or most preferably at least 97% identical to the sequences shown in SEQ ID NO:3, SEQ ID NO:19, or to the phytase encoding sequence comprised in a cell of DSM 13352.

As is discussed elsewhere herein, there are a number of ways to introduce a heterologous DNA sequence into a host cell known in the art.

A further embodiment relates to a Pichia cell of the first aspect, wherein the nucleotide sequence is present on an extrachromosomal DNA construct, preferably a plasmid, or more preferably the nucleotide sequence is integrated in the cell's chromosome, preferably in multiple copies.

Also well known in the art are ways of expressing heterologous DNA sequences encoding polypeptides of interest in host cells. Several promoters have been described to work well in Pichia cells as described above.

Accordingly a preferred embodiment of the invention relates to a Pichia cell of the first aspect, wherein the nucleotide sequence is transcribed from a promoter of a methanol-inducible P. methanolica gene, preferably the promoter is a native promoter of a P. methanolica gene encoding an enzyme selected from the group consisting of alcohol oxidase, dihydroxyacetone synthase, formate dehydrogenase, and catalase.

For industrial production of polypeptides it is of considerable interest to have the polypeptides secreted from the producing host cells in order to minimize costs of isolating the polypeptide, ways of promoting efficient secretion are well known in the art. A polypeptide may be efficiently secreted if it carries an effective secretion signal peptide recognized by the host cell's secretion apparatus. A number of secretion signal peptides have been described to work well in yeast host cells as described elsewhere herein. The present inventors found that even without the addition of a well-characterized yeast secretion signal peptide, efficient secretion could be achieved from a host cell of a polypeptide carrying its native original secretion signal sequence.

A preferred embodiment of the invention relates to a Pichia cell of the first aspect, wherein the polypeptide comprises its native original secretion signal sequence.

The present invention relies on adjusting the codon usage of an isolated DNA sequence to match the preferred codon usage of Pichia methanolica. An isolated DNA construct comprising at least one copy of a nucleotide sequence encoding a polypeptide heterologous to Pichia methanolica, wherein the codon usage of said sequence has been adjusted to match the preferred codon usage of P. methanolica, as defined herein.

A preferred embodiment of the invention relates to an isolated DNA construct of the second aspect of the invention, wherein the nucleotide sequence encodes an enzyme, preferably the nucleotide sequence encodes an enzyme having phytase or xylanase activity, and more preferably the nucleotide sequence comprises a DNA sequence at least 90% identical, or preferably at least 93% identical, or more preferably at least 95% identical, or most preferably at least 97% identical to the sequences shown in SEQ ID NO:3, SEQ ID NO:19, or to the phytase encoding sequence comprised in a cell of DSM 13352.

Another preferred embodiment of the invention relates to an isolated DNA construct of the second aspect of the invention, wherein the nucleotide sequence is transcribed from a promoter of a methanol-inducible P. methanolica gene, preferably the promoter is a native promoter of a P. methanolica gene encoding an enzyme selected from the group consisting of alcohol oxidase, dihydroxyacetone synthase, formate dehydrogenase, and catalase. Other promoters generally recognized as working well in Pichia cells are those of the glycolytic enzymes, such as glyceraldehyde-3-phosphate dehydrogenase, triose phosphate isomerase, enolase etc.

Further a preferred embodiment of the invention relates to an isolated DNA construct of the second aspect of the invention, wherein the nucleotide sequence encodes a polypeptide comprising its native original secretion signal sequence.

As mentioned previously the invention relates to industrial production, i.e. a method of producing a polypeptide of interest in a Pichia cell, where the polypeptide is encoded by a nucleotide sequence heterologous to P. methanolica, said method comprising the steps of:

(a) adjusting the codon usage of the sequence to match the preferred codon usage of P. methanolica, as defined herein; and

(b) cultivating a Pichia cell comprising at least one copy of the codon usage adjusted sequence of step a) under appropriate growth conditions to express the sequence and achieve production of the polypeptide of interest.

A preferred embodiment of the invention relates to a method of the third aspect, wherein the Pichia cell is a Pichia methanolica cell.

Yet another preferred embodiment of the invention relates to a method of the third aspect, wherein the nucleotide sequence encodes an enzyme, preferably the nucleotide sequence encodes an enzyme having phytase or xylanase activity, or more preferably the nucleotide sequence comprises a DNA sequence at least 90% identical, or preferably at least 93% identical, or more preferably at least 95% identical, or most preferably at least 97% identical to the sequences shown in SEQ ID NO:3, SEQ ID NO:, or to the phytase encoding sequence comprised in a cell of DSM 13352.

The present inventors succeeded in obtaining much improved yields of polypeptides of interest by using a method of the third aspect of the invention, in one non-limiting example they even obtained a yield that exceeded 10 times the yield obtained with the conventional method.

Consequently a preferred embodiment relates to a method of the third aspect, wherein the polypeptide is produced in a yield at least three times higher than what is obtained from an otherwise identical cell comprising a sequence not adjusted to the preferred codon usage of P. methanolica, as defined herein, preferably at least five times higher, more preferably seven, even more preferably eight, yet more preferably nine, and most preferably ten times higher than what is obtained from an otherwise identical cell comprising a sequence not adjusted to the preferred codon usage of P. methanolica, as defined herein.

In parallel with the two first aspects, preferred embodiments relate to methods of the third aspect, wherein the nucleotide sequence is transcribed from a promoter of a methanol-inducible P. methanolica gene, preferably the promoter is a native promoter of a P. methanolica gene encoding an enzyme selected from the group consisting of alcohol oxidase, dihydroxyacetone synthase, formate dehydrogenase, and catalase; and preferably methanol is added to the cell during the cultivating step, whereby the methanol-inducible promoter is induced; it is also preferred that the nucleotide sequence encodes a polypeptide comprising its native original secretion signal sequence.

Material and Methods

Purchased Material (E. coli, Plasmid and Kits)

E. coli DH12S (Gibco BRL) and E. coli JM109 (Toyobo) were used for plasmid construction and amplification. Amplified plasmid was recovered with Qiagen® Plasmid Kit (Qiagen). Ligation was done with DNA ligation kit (Takara) or T4 DNA ligase (Boehringer Mannheim). Polymerase Chain Reaction (PCR) was carried out either with Taq DNA polymerase (Boehringer Mannheim) or with Expand™ PCR system especially Expand™ Long Template (Boehringer Mannheim). QIAquick™ Gel Extraction Kit (Qiagen) was used for the purification of PCR fragments and extraction of DNA fragment from agarose gel. A plasmid pT7blue T-vector (Novagen) was used for the cloning of PCR fragments.

Expression Host Strain

The expression host strain is a Pichia methanolica PMAD16 (ade2-pep4Δ prb1Δ) described in WO 99/14347. It is a vacuolar protease deficient mutant originally generated from P. methanolica CBS 6515. Two major vacuolar proteinases, i.e. Proteinase A encoded by the pep4 gene and Proteinase B encoded by the prb1 gene, are inactivated by disrupting these genes (WO 99/14347).

Expression Plasmid

The expression cassette plasmid pCZR134 is described in WO 99/14347. It comprises the promoter and terminator of the alcohol oxidase gene AUG1 from P. methanolica (WO 99/14347) and the ADE2 gene encoding phosphoribosyl-5-aminoimidazole carboxylase (WO 99/14347) from P. methanolica.

Another expression cassette plasmid is pGAP, which is identical to pCZR134 but contains the promoter of the gene encoding the glyceraldehyde-3-phophate dehydrogenase GAP1 from P. methanolica (described in WO 00/56903, SEQ ID NO:8 therein) instead of the AUG1 promoter as described above.

Transformation of P. methanolica

Transformation of Pichia methanolica can be done using the general methods for yeast transformation. As the most preferred method, the procedure of electro transformation is described below. The P. methanolica host strain is grown in non-selective YPD medium (20 g/l of Pepton, Difco, 10 g/l of yeast extract, Difco and 20 g/l of glucose) at 37° C. for overnight with vigorous aeration, and the culture is diluted 100-fold in 200 ml fresh, pre-warned YPD. This is further cultivated at 37° C. until the optical density at 663 nm becomes 1.2-1.5, which corresponds to 9×10⁷ cells/ml. Cells are collected by centrifugation at 3000 g for 10 minutes, and re-suspended in 40 ml of 50 mM potassium phosphate buffer pH 7.5 containing 25 mM of dithiothreitol (DTT), then incubated at 37° C. for 15 minutes. The cells are washed twice with ice-cold electroporation buffer STM (270 mM sucrose, 10 mM Tris pH 7.5 and 1 mM CaCl2), at first with 200 ml (the original culture volume), then with 100 ml (50% original volume). Finally the cells are re-suspended in 1 ml (0.5% of original volume) ice-cold STM buffer to a concentration of approximately 2×10¹⁰ electrocompetent cells/ml.

0.1-10 μg of DNA (volume <10 μl) is added to 100 μl of electrocompetent cells and placed into an ice-cold electroporation cuvette with an electrode gap of 2 mm. A pulsed electric field is provided at 3.75 kV/cm for 20 millisecond, using a BioRad Gene Pulser™ electroporator on which resistance is set to >600 Ω or “infitine” and capacitance is set to 25 μF. After delivering the pulse, 1 ml of YPD is added to the cell/DNA mixture and incubated for 1 hour at 30° C.

Cells are harvested by centrifugation at 3000 g for 5 minutes, then washed with 1 ml of yeast nitrogen base (6.7 g/l yeast nitrogen base without amino acids, Difco), and plated on minimal selective media, e.g. SC glucose. Host cells having an ade2 mutation appear as pink colonies on minimal medium, whereas transformants that acquired a stable integration (Ade+) can grow rapidly as white colonies on minimal medium.

### EXAMPLE 1

A cDNA Clone of the Thermomyces Xylanase Gene

The cDNA sequence of the Thermomyces lanuginosus DSM4109 xylanase gene was published (WO 96/32472). For this study we obtained A Saccharomyces cerevisiae strain, DSM 10133, containing a plasmid comprising the full-length xylanase cDNA of the Thermomyces lanuginosus DSM4109.

Construction of Expression Plasmid pCaXI1

The Thermomyces xylanase cDNA was amplified by PCR using primers xyN and xyC to introduce desirable restriction enzyme cleavage sites at both ends of the PCR fragment:

xyN (SEQ ID NO:1): CCGGAATTCATGGTCGGCTTTACCCCCGTT.
XyC (SEQ ID NO:2): AAGGAAAAAAGCGGCCGCACTAGTTTAGCCCACGTCAGCAAC.

The PCR reaction mixture comprised 50 ng of template cDNA, 0.05 mM of dNTPs, 100 pmol of each primer and lunit of Taq polymerase in 50 μl of Taq buffer with MgCl2 (Boehringer Mannheim). The reaction was initiated at 94° C. for 3.5 minutes followed by 30 cycles of 94° C. for 1.5 minutes, 50° C. for 1.5 minutes and 72° C. for 3 minutes, with the final extension at 72° C. for 10 minutes. The amplified 0.7 kb fragment was digested with EcoRI and SpeI, and purified on 1.5% agarose gel. Obtained fragment was subjected to the ligation with pCZR134 linearized by EcoRI and SpeI. The resulting plasmid was termed pCaXI1.

Gene Synthesis of Themomyces Xylanase Gene

The Themomyces xylanase gene was synthesized to change the codon usage to have A or T as the preferred third letter of each codon. This is not a full optimization to the preferred codon usage of Pichia methanolica, as defined herein, but rather a first attempt to see if there is any advantage in optimizing the codons further. Two fragments XyC12 and XyC37 were synthesized by two-step PCR. The resulting designed xylanase encoding sequence is shown as SEQ ID NO:3.

Oligonucleotides Used for the First PCR Reaction Were as Follows:

For the synthesis of XyC12:

| | | |
|---|---|---|
| xyC1 | (SEQ ID NO:4): | atggttggttttactccagttgctttagctgctttagctgctactggtgctttagctttc |
| | | ccagcaggtaatgctactgaattagaaaaaagacaaacaactccaaattctgaaggttgg. |
| xyC2r | (SEQ ID NO:5): | cataggtaccaccttctaaattagtataagtagcttgagcaccaccatca |
| | | gaccaccaagaataataataaccatcatgccaaccttcagaatttggaga. |

For the synthesis of XyC37:

| | | |
|---|---|---|
| xyC3 | (SEQ ID NO:6): | ggtacctatgaaatttcttggggtgatggtggtaatttagttggtggtaaaggttggaat |
| | | ccaggtttaaatgcaagagctattcattttgaaggtgtttatcaaccaaatggtaattct. |
| xyC4r | (SEQ ID NO:7): | ctagtagttttacctaatctataaatagaaccatcacattcaacagtacctaaatcag |
| | | tagcaccagaagatggatcataagaccaaaattttcaacaatataatattcaactaatgga |
| xyC5 | (SEQ ID NO:8): | ttctggtgctactgatttaggtactgttgaatgtgatggttctatttatagattaggtaa |
| | | aactactagagttaatgcaccatctattgatggtactcaaactttcgatcaatattggtc. |
| xyC6r | (SEQ ID NO:9): | taataatgatcaccattaacattcaaaccagctctagcccaagcatcgaaatgac |
| | | aaccagtttgaacagtaccagaagttcttttatcttgtctaacagaccaatattgatcgaaagtt. |
| xyC7 | (SEQ ID NO:10): | ttaatggtgatcattattatcaaattgttgcaactgaaggttatttctcttctggttatg |
| | | ctagaattactgttgctgatgttggttaa. |

Oligonucleotides Used for the Second PCR Reaction Were as Follows:

For the synthesis of XyC12:
xyc12N (SEQ ID NO:11): ataagaatgcggccgcatggttggttttactccagt.
xyc12C (SEQ ID NO:12): tcataggtaccaccttctaaattagtataa.
For the synthesis of XyC37:
xyc37N (SEQ ID NO:13): ttagaaggtggtacctatgaaatttcttggggtgat.
xyc37C (SEQ ID NO:14): ttttccttttgcggccgcttaaccaacatcagcaac.

The first PCR reaction mixture contained 1 pmol of each oligonucleotide, 0.35 mM dNTP, 2.6 units of Expand™ polymerase in 50 μl of provided buffer. Reaction started at 94° C. for 1 minute followed by 6 cycles of 94° C. for 0.5 minutes, 50° C. for 5 minutes and 72° C. for 1 minute with the final extension at 72° C. for 10 minutes. Five μl of resulting first PCR reaction solution was used for the second PCR with 0.35 mM dNTP, 100 pmol of each oligonucleotide and 2.6 units of Expand™ polymerase in 50 μl of provided buffer. The reaction was carried out at 94° C. for 3.5 minutes first followed by 30 cycles of 94° C. for 1.5 minutes, 50° C. for 1.5 minutes and 72° C. for 3 minutes with the final extension at 72° C. for 10 minutes. The amplified fragments were purified on 4% agarose gel then ligated into pT7blue T-vector resulting pTXyC12-7 and pTXyC37-715 respectively. They were confirmed to have no PCR error by sequencing. A KpnI fragment of around 200bp was isolated from pTXyC12-7 then ligated with a 3.7 kb of KpnI fragment from pTXyC37-715. The resulting plasmid pTXyC17-1 comprises the sequence of the codon-optimized synthesized Themomyces xylanase gene (SEQ ID NO:3).

Construction of Expression Plasmid pCaXS3

The synthesized Thermomyces xylanase gene was amplified by PCR using the following primers;

a)
(SEQ ID NO:15): CCGGAATTCATGGTTGGTTTTACTCCAGTTGCT.
b)
(SEQ ID NO:16): GGACTAGTTTAACCAACATCAGCAACAGTAAT.

The reaction mixture comprised 10 ng of synthesized xylanase gene, 0.35 mM of dNTP, 100 pmol of each primer and 2.6 units of Expand™ polymerase in 50 μl of provided buffer with MgCl2. The PCR reaction was initiated at 94° C. for 3.5 minutes followed by 30 cycles of 94° C. for 1.5 minutes, 55° C. for 1.5 minutes and 68° C. for 3 minutes with the final extension at 72° C. for 10 minutes. The amplified gene was digested with EcoRI and SpeI, and ligated into pCZR134 cut by EcoRI and SpeI beforehand and the resulting plasmid was termed pCaXS3.

Transformation of P. methanolica with pCaXI1 and pCaXS3

The expression plasmids pCaXI1 and pCaXS3 were digested with NotI and 6.2 kb fragments were used for the transformation of P. methanolica PMAD16 to achieve a Ade+ phenotype. Transformed cells were grown on SC glucose (2% glucose, 0.01% tryptophan, 0.02% threonine, 0.5% casamino acid, in basal salt without amino acids (per liter, 6.68 g yeast nitrogen base w/o amino acid (Difco), 10 g succinic acid, 6 g NaOH, and 2% Agar Noble (Difco) containing 1.2 M sorbitol). Rapidly growing white colonies were isolated as stable transformants.

Obtained transformants were grown on YPM agar (20 g/l of Pepton, Difco, 10 g/l of yeast extract, Difco, 20 g/l of Agar Noble, Difco and 20 ml/l of methanol) containing 0.1% AZCL™-xylan (Megazyme™) for 3-5 days at 30° C. Positive transformants were identified as the colonies surrounded by a blue halo.

Xylanase producing cells were inoculated in YPD medium (20 g/l of Pepton, Difco, 10 g/l of yeast extract, Difco and 20 g/l of glucose) in a 500 ml shaking flask, and cultivated under vigorous aeration overnight at 30° C. One ml of methanol was added to each shaking flask and it was cultivated one more day. Cells were removed by centrifugation and xylanase activity in the supernatant was measured using 0.5% Azo-Wheat Arabinoxylan (Megazyme™) as substrate. The measurement reaction was employed at 30° C., pH 6 for 30 minutes and released colour was measured as the absorbance at 585 nm. The amount of enzyme protein was estimated from the activity. The results are shown below; our conclusion to this first attempt is that the codon optimization did indeed have a significant positive effect on the obtainable yield from a Pichia cell. However, since we did not fully optimize the codons of the xylanase encoding sequence to match the preferred codon usage of P. methanolica, it may be possible to obtain even greater yields by adjusting the sequence thoroughly.

| Strain | Plasmid | Gene | Yield in shaking flask |
|---|---|---|---|
| PX1242 | pCaXI1 | intact gene | 12 mg/l |
| PXS5 | pCaXS3 | synthesized gene | 20 mg/l |

A transformant carrying the synthesized Thermomyces xylanase gene was inoculated in two flasks with 110 ml of YEPD (10 g/l Bacto yeast extract, 20 g/l Bacto peptone and 20 g/l glucose) in 500 ml shaking flasks then cultivated under vigorous aeration condition at 30° C. for 16-18 hours. Most of the cells were used for the subsequent inoculation of the fermentor. The fermentation vessel was filled with 2 liters of media containing 43 g (NH4)2SO4, 5.2 g K2HPO4, 19 g KH2PO4, 0.8 g FeSO4.7H2O, 2.3 g sodium citrate with 2aq. After autoclaving and cooling down to 30° C., 94 ml of 60% glucose, 40 ml of 1 M MgSO4.7H2O, 4 ml of 1 M CaCl2, 40 ml of trace metal solution (containing, per litter, 27.8 g Fe SO4.7H2O, 0.5 g CuSO4.5H2O, 1.09 g ZnCl2, 1.35 g MnSO4.H2O, 0.48 g CoCl2.6H2O, 0.24 g Na2MoO4.2 H2O, 0.5 g H3BO3, 0.08 g KI, 0.26 g NiSO4.6 H2O, 0.56 g thiamine hydrochloride, 5 mg biotin, 1 ml H2SO4) and 4 ml of vitamin solution (containing, per litter, 47 g inositol, 25 g calcium pantothenate, 1.3 g pyridoxine hydrochloride, 5.6 g thiamine hydrochloride, 0.1 g biotin) were added. The pH of the fermentation media was adjusted to 5.0 and during the fermentation pH was controlled automatically with 25% ammonia water and 10% H₃PO₄. Aeration was provided as compressed air at a flow rate of 3 liters per minute initially and agitation at the rate of 100 rpm. After inoculation the agitation was controlled automatically from 100 rpm to 1000 rpm to keep the dissolved oxygen level at minimum 30% of saturation. Following exhaustion of glucose after inoculation, a glucose feed was initiated at 12 hours at the rate of 0.27% glucose/hour. The feed was increased stepwise to 0.38% glucose/hour at 18 hours and to 0.5% glucose/hour at 30 hours. After 40 hours, methanol induction of the AUG1 promoter was started by methanol feed at the rate of 0.3% methanol/hour, in parallel the glucose feed rate was reduced to 0.38% glucose/hour. Finally mixed feeding (0.6% methanol/hour, 0.27% glucose/hour) was run for the rest of fermentation. Increasing methanol feeding elevated the enzyme productivity. The yield of Thermomyces xylanase was calculated to be 750 mg/l with the 110 g/kg of dried cells at 170 hours.

### EXAMPLE 2

cDNA Clone of A. fumigatus Phytase Gene

The sequence of the phytase gene from Aspergillus fumigatus ATCC 13073 was published previously (EP 0897010). This phytase contains an amino acid mutation Q27L, and the phytase of the invention comprises a further mutation at the second amino acid, V21, due to the introduction of a restriction enzyme cleavage site around the translation initiation codon.

Construction of Expression Plasmid pCaPhy3

A cDNA of A. fumigatus phytase gene was amplified by PCR using the primers shown below;

| | |
|---|---|
| A1) (SEQ ID NO:17): | CCGGAATTCATGATTACTCTGACTTTCCTGCTTTCGGCGG |
| | CGTATCTGCTTTCTGGTAGAGTGTCTGCGGCACCTAGTTCTGCTGGCTC. |
| B1) (SEQ ID NO:18): | CTAGACTAGTCTAGTCAACTAAAGCACTCTCCCCA. |

The reaction mixture comprising 10 ng of cDNA clone of A. fumigatus phytase, 0.35 mM dNTPs, 100 pmol of each primers and 2.6 units of Expand™ polymerase in 50 μl of provided buffer with MgCl2, was incubated at 94° C. for 2 minutes followed by 10 cycles of 94° C. for 10 seconds, 55° C. for 30 seconds, 68° C. for 45 seconds. After that another 20 cycles were performed as 94° C. for 10 seconds, 55° C. for 30 seconds, 68° C. for 45 plus n×20 seconds (n: cycle number).

The final extension was at 68° C. for 7 minutes. Amplified 1.3 kb fragment was purified on 1.2% agarose gel and digestion with EcoRI and SpeI. It was used for the ligation with pCZR134 digested with the same restriction enzymes and the obtained plasmid was termed pCaPhy3.

Construction of the Expression Plasmid pCPS40

DNA encoding the A. fumigatus phytase was synthesized, the sequence was designed based on the codon usage of two genes from Pichia methanolica, i.e. ADE1 and AUG1. Codon usage of designed sequence is shown in Table 1.

This designed codon optimized phytase DNA sequence is shown in (SEQ ID NO:19).

TABLE 1

| | | P. | P. | | |
|---|---|---|---|---|---|
| Amino | | methanolica | methanolica | Original | Optimized |
| acid | Codon | ADE1 | AUG1 | phytase gene | phytase gene |
| F | TTT | 9 | 10 | 12 | 12 |
| | TTC | 6 | 19 | 15 | 15 |
| L | TTA | 17 | 16 | 0 | 31 |
| | TTG | 14 | 19 | 10 | 10 |
| | CTT | 1 | 2 | 7 | 0 |
| | CTC | 2 | 0 | 5 | 0 |
| | CTA | 7 | 3 | 4 | 4 |
| | CTG | 2 | 0 | 19 | 0 |
| I | ATT | 19 | 2 | 8 | 9 |
| | ATC | 18 | 12 | 6 | 6 |
| | ATA | 5 | 0 | 1 | 0 |
| M | ATG | 17 | 17 | 6 | 6 |
| V | GTT | 29 | 36 | 6 | 25 |
| | GTC | 12 | 4 | 4 | 4 |
| | GTA | 0 | 0 | 3 | 0 |
| | GTG | 3 | 0 | 16 | 0 |
| S | TCT | 10 | 20 | 6 | 15 |
| | TCC | 5 | 10 | 10 | 17 |
| | TCA | 9 | 11 | 3 | 16 |
| | TCG | 4 | 0 | 13 | 0 |
| | AGT | 2 | 0 | 9 | 0 |
| | AGC | 3 | 0 | 7 | 0 |
| P | CCT | 7 | 1 | 7 | 7 |
| | CCC | 1 | 0 | 4 | 0 |
| | CCA | 19 | 44 | 4 | 15 |
| | CCG | 5 | 0 | 7 | 0 |
| T | ACT | 22 | 31 | 11 | 21 |
| | ACC | 6 | 11 | 9 | 9 |
| | ACA | 4 | 2 | 1 | 1 |
| | ACG | 2 | 0 | 10 | 0 |
| A | GCT | 27 | 40 | 9 | 25 |
| | GCC | 14 | 7 | 12 | 18 |
| | GCA | 7 | 1 | 6 | 0 |
| | GCG | 3 | 0 | 16 | 0 |
| Y | TAT | 5 | 9 | 4 | 4 |
| | TAC | 9 | 20 | 14 | 14 |
| H | CAT | 8 | 4 | 4 | 4 |
| | CAC | 7 | 18 | 4 | 4 |
| Q | CAA | 18 | 12 | 2 | 15 |
| | CAG | 2 | 0 | 13 | 0 |
| N | AAT | 11 | 2 | 6 | 0 |
| | AAC | 13 | 30 | 13 | 19 |
| K | AAA | 15 | 7 | 1 | 1 |
| | AAG | 17 | 31 | 25 | 25 |
| D | GAT | 15 | 30 | 8 | 8 |
| | GAC | 13 | 18 | 17 | 17 |
| E | GAA | 20 | 33 | 4 | 20 |
| | GAG | 7 | 3 | 16 | 0 |
| C | TGT | 9 | 11 | 2 | 10 |
| | TGC | 1 | 2 | 8 | 0 |
| W | TGG | 2 | 10 | 5 | 5 |
| R | CGT | 2 | 1 | 1 | 1 |
| | CGC | 0 | 0 | 7 | 0 |
| | CGA | 0 | 0 | 3 | 0 |
| | CGG | 0 | 0 | 6 | 0 |
| | AGA | 14 | 31 | 2 | 18 |
| | AGG | 2 | 0 | 1 | 1 |
| G | GGT | 26 | 53 | 3 | 33 |
| | GGC | 11 | 1 | 16 | 0 |
| | GGA | 3 | 0 | 6 | 0 |
| | GGG | 2 | 0 | 8 | 0 |
| Stop | TAA | 0 | 0 | 0 | 0 |
| | TAG | 0 | 1 | 0 | 0 |
| | TGA | 1 | 0 | 1 | 1 |
| Total | | 544 | 665 | 66 | 466 |

Seventy oligonucleotides (SEQ ID NOS:20-89) were prepared for the construction of the codon optimized phytase gene, and subjected to PCR using Expand™ Long Template (Boehringer Mannheim) for assembly. The reaction mixture comprised 125 pmol of each of the seventy oligonucleotides shown in SEQ ID NOS:20-89, 0.2 mM dNTPs and 2.6 units of Expand™ polymerase in 50 μl of provided buffer. The reaction was employed as 55 cycles of 94° C. for 30 seconds, 52° C. for 30 seconds and 68° C. for 30 seconds. The second PCR was carried out using the resulting first reaction mixture as template and the following set of primers:

c) (SEQ ID NO:90): CGGAATTCATGATTACTTTAACTTTCTTATTATCAGCTTATTTA.
d) (SEQ ID NO:91): GGACTAGTTCAAGAAAAACATTCACCCC.

The reaction mixtures comprised 0.5 μl, 1 μl or 2 μl of the first PCR mixture, 30 pmol of each primer, 0.2 mM dNTPs and 2.6 units of Expand TM polymerase in 50 μl of provided buffer. The reaction was employed as 25 cycles of 94° C. for 30 seconds, 50° C. for 30 seconds and 68° C. for 60+10×n seconds (n: cycle number). An amplified 1.3 kb fragment was purified with QIAquick and cloned into pT7blue T-vector. Two independent plasmids pTPhy5 and pTPhy8 were selected for further construction, they had a couple of PCR generated sequence errors within the HindIII-SpeI region of pTPhy5 and the EcoRI-HindIII region of pTPhy8.

Each plasmid was digested with HindIII, and a 3.4 kg fragment from pTPhy5 and a 0.7 kb fragment from pTPhy8 were isolated and ligated together, resulting in the plasmid pTPhy58, which comprises the entire sequence of the synthetic A. fumigatus phytase gene.

The plasmid pTPhy58 was digested with EcoRI and SpeI, and a 1.3 kb fragment was isolated and ligated into the expression vector pCZR134. The resulting phytase expression plasmid was designated pCPS40.

The entire sequence of the synthetic codon optimized A. fumigatus phytase gene (SEQ ID NO:19) is comprised in the strain E. coli NN049526, which was deposited with DSMZ Mar. 1, 2000 with the accession number DSM 13352. The deposit was made by Novo Nordisk A/S and was later assigned to Novozymes A/S.

Transformation of P. methanolica with pCaPhy3 and pCPS40

The expression plasmids pCaPhy3 and pCPS40 were digested with NotI and 6.7 kb fragments were used for the transformation of P. methanolica PMAD16. Transformed cells were grown on minimal medium SC glucose containing 1.2 M sorbitol. White colonies growing rapidly were isolated as stable transformants. The obtained transformants were subsequently grown on YPM agar containing 0.1% phytic acid (Sigma P8810) at 30° C. overnight, then 0.1 M CaCl2 was poured onto the grown agar plate to detect phytase production. Positive transformants were detected as colonies surrounded by a clear halo.

Phytase producing strains were inoculated in 50 ml of YPD medium in 500 ml of shaking flasks and cultivated at 30° C. with vigorous aeration. After overnight cultivation, 1 ml of methanol was added to each shaking flask and the cultivation was continued one more day. Cells were removed by centrifugation and phytase activity in the supernatant was measured using 0.15% sodium phytate in 100 mM sodium acetate buffer pH 5.5 as the substrate. The reaction was carried out at room temperature for 15 minutes, and liberated phosphate ions were quantified by colorization with 1% ammonum molybdate, 0.26 M FeSO4 in 3.2% H2SO4 and absorbance was measured at 650 nm. The amount of enzyme protein was calculated from the activity based on the known specific activity per mg enzyme protein. The results are shown in Table 2.

TABLE 2

| Strain | Plasmid | Used gene | Yield in shaking flask |
|---|---|---|---|
| PPi 51 | pCaPhy3 | cDNA | 1 mg/l |
| PPs237 | pCPS40 | codon optimized | 20 mg/l |

The transformants mentioned above were cultivated as described in example 1. The yield of A. fumigatus phytase from a transformant generated with pCaPhy3, carrying non codon optimized cDNA of the phytase gene, was calculated to be 310 mg/l with 120 g/kg of dried cells at 170 hours of fermentation, whereas the yield from the transformant with a codon-optimised gene was estimated to be 3.5 g/l with 115 g/kg of dried cells at 190 hours of fermentation.

Thus the yield from the codon optimized transformant was approximately 10 times higher than what could be obtained from the transformant carrying the original cDNA phytase gene.

### EXAMPLE 3

Expression of the A. fumigatus phytase from the GAP-Promoter

A synthesized A. fumigatus phytase gene was obtained from pCPS40 as described in Example 2 by digesting with the restriction enzymes EcoRI and SpeI. The isolated 1.3 kb fragment was ligated into the pGAP vector, which was initially pre-digested with the same enzymes, the resulting plasmid was termed pGAPphy. The plasmid pGAPphy was then digested with NotI and the 6.9 kb fragment was used for the transformation of P. methanolica PMAD16. Transformants were isolated as described in Example 2.

Obtained transformants were cultivated in 5 ml of YPD medium at 30° C. overnight with vigorous aeration, then 1 ml of grown culture was transferred to fresh 100 ml of YPD medium in a shaking flask. After one-day cultivation at 30° C., 2 ml of 20% glucose solution was added to each flask and cultivation was continued for one more day. Enzyme activity of the culture broth, from which the grown cells were removed, was measured to estimate the enzyme productivity. The estimated phytase productivity was 47 mg/l.

A transformant carrying a synthesized Aspergillus fumigatus phytase gene was fermented in 5 L jar-fermenter. The fermentation vessel was filled with 2 liters of medium containing 43 g (NH4)2SO4, 5.2 g K2HPO4, 19 g KH2PO4, 0.8 g Fe SO4.7H2O, 2.3 g sodium citrate with 2aq. After autoclaving and cooling down to 30° C., 94 ml of 60% glucose, 40 ml of 1 M MgSO4.7H2O, 4 ml of 1 M CaCl2, 40 ml of trace metal solution and 4 ml of vitamin solution were added. The pH of fermentation media was adjusted to 5.0 and during the fermentation it was controlled automatically above 5.0 with 25% ammonia water. Aeration was provided as compressed air at a flow rate of 3 liters per minute initially and agitation at the rate of 100 rpm. After inoculation the agitation was controlled automatically from 100 rpm to 1000 rpm to keep the dissolved oxygen level at minimum 30% of saturation. Around 11 hours after inoculation, 1100 ml of 60% glucose was supplemented. The yield of Aspergillus fumigatus phytase at 50 hours was calculated to 1000 mg/l with the 65 g/kg of dried cells.

1. A Pichia cell comprising at least one copy of a heterologous nucleotide sequence encoding a polypeptide of interest, wherein the codon usage of said sequence has been adjusted to match the preferred codon usage of P. methanolica, as defined herein.

2. The cell of claim 1, which is a Pichia methanolica cell.

3. The cell of claim 1, wherein the nucleotide sequence encodes an enzyme.

4. The cell of claim 1, wherein the nucleotide sequence encodes an enzyme having phytase or xylanase activity.

5. The cell of claim 1, wherein the nucleotide sequence comprises a DNA sequence at least 90% identical to the sequence shown in SEQ ID NO:3 or SEQ ID NO:19, or to the phytase encoding sequence contained in a cell of DSM 13352.

6. The cell of claim 1, wherein the nucleotide sequence is present on an extrachromosomal DNA construct, preferably a plasmid.

7. The cell of claim 1, wherein the nucleotide sequence is integrated in the cell's chromosome.

8. The cell of claim 1, wherein the nucleotide sequence is transcribed from a promoter of a methanol-inducible P. methanolica gene.

9. The cell of claim 8, wherein the promoter is a native promoter of a P. methanolica gene encoding an enzyme selected from the group consisting of alcohol oxidase, dihydroxyacetone synthase, formate dehydrogenase, and catalase.

10. The cell of claim 1, wherein the polypeptide comprises its native original secretion signal sequence.

11. An isolated DNA construct comprising at least one copy of a nucleotide sequence encoding a polypeptide heterologous to Pichia methanolica, wherein the codon usage of said sequence has been adjusted to match the preferred codon usage of P. methanolica, as defined herein.

12. The DNA construct of claim 11, wherein the nucleotide sequence encodes an enzyme.

13. The DNA construct of claim 12, wherein the enzyme has phytase or xylanase activity.

14. The DNA construct of claim 11, wherein the nucleotide sequence comprises a DNA sequence at least 90% identical to the sequence shown in SEQ ID NO:3 or SEQ ID NO:19, or to the phytase encoding sequence contained in a cell of DSM 13352.

15. The DNA construct of claim 11, wherein the nucleotide sequence is transcribed from a promoter of a methanol-inducible P. methanolica gene.

16. The DNA construct of claim 15, wherein the promoter is a native promoter of a P. methanolica gene encoding an enzyme selected from the group consisting of alcohol oxidase, dihydroxyacetone synthase, formate dehydrogenase, and catalase.

17. The DNA construct of claim 11, wherein the nucleotide sequence encodes a polypeptide comprising its native original secretion signal sequence.

18. A method of producing a polypeptide of interest in a Pichia cell, wherein the polypeptide is encoded by a nucleotide sequence heterologous to P. methanolica, comprising the steps of:
(a) adjusting the codon usage of the sequence to match the preferred codon usage of P. methanolica, as defined herein; and
(b) cultivating a Pichia cell comprising at least one copy of the codon usage adjusted sequence of step (a) under appropriate growth conditions to express the sequence and achieve production of the polypeptide of interest.

19. The method of claim 18, wherein the Pichia cell is a Pichia methanolica cell.

20. The method of claim 18, wherein the nucleotide sequence encodes an enzyme.

21. The method of claim 20, wherein the enzyme has phytase or xylanase activity.

22. The method of claim 18, wherein the nucleotide sequence comprises a DNA sequence at least 90% identical to the sequences shown in SEQ ID NO:3 or SEQ ID NO:19, or to the phytase encoding sequence contained in a cell of DSM 13352.

23. The method of claim 18, wherein the polypeptide is produced in a yield that is at least three times higher than what is obtained from an otherwise identical cell comprising a sequence not adjusted to the preferred codon usage of P. methanolica, as defined herein.

24. The method of claim 23, wherein the polypeptide is produced in a yield that is at least five times higher than what is obtained from an otherwise identical cell comprising a sequence not adjusted to the preferred codon usage of P. methanolica, as defined herein.

25. The method of claim 24, wherein the polypeptide is produced in a yield that is seven times higher than what is obtained from an otherwise identical cell comprising a sequence not adjusted to the preferred codon usage of P. methanolica, as defined herein.

26. The method of claim 25, wherein the polypeptide is produced in a yield that is eight times higher than what is obtained from an otherwise identical cell comprising a sequence not adjusted to the preferred codon usage of P. methanolica, as defined herein.

27. The method of claim 26, wherein the polypeptide is produced in a yield that is nine times higher than what is obtained from an otherwise identical cell comprising a sequence not adjusted to the preferred codon usage of P. methanolica, as defined herein.

28. The method of claim 27, wherein the polypeptide is produced in a yield that is ten times higher than what is obtained from an otherwise identical cell comprising a sequence not adjusted to the preferred codon usage of P. methanolica, as defined herein.

29. The method of claim 18, wherein the nucleotide sequence is transcribed from a promoter of a methanol-inducible P. methanolica gene.

30. The method of claim 29, wherein the promoter is a native promoter of a P. methanolica gene encoding an enzyme selected from the group consisting of alcohol oxidase, dihydroxyacetone synthase, formate dehydrogenase, and catalase.

31. The method of claim 29, wherein methanol is added to the cell during the cultivating step, whereby the methanol-inducible promoter is induced.

32. The method of claim 18, wherein the nucleotide sequence encodes a polypeptide comprising its native original secretion signal sequence.
